(19) United States
(12) Patent Application Publication
(10) Pub. No.: US 2021/0393136 A1
(43) Pub. Date: Dec. 23, 2021

(54) INTRAORAL MEASUREMENT DEVICE

(71) Applicant: Konica Minolta, Inc., Tokyo (JP)

(72) Inventors: Yoshihiro INAGAKI, Tokyo (JP); Atsushi NAGAOKA, Okazaki-shi (JP)

(73) Assignee: Konica Minolta, Inc., Tokyo (JP)

(21) Appl. No.: 17/352,826

(22) Filed: Jun. 21, 2021

(30) Foreign Application Priority Data
Jun. 22, 2020 (JP) 2020-106693

(51) Int. Cl. A61B 5/00; A61C 19/04

(52) U.S. Cl. CPC A61B 5/0088; A61B 2562/0238; A61C 19/04; A61B 5/0073

(57) ABSTRACT

An intraoral measurement device includes a measurement optical system that includes: a laser light source; a time-of-flight (TOF) sensor; and a lens. The laser light source irradiates a measuring region including a measuring target with laser light that is intensity-modulated in synchronization with the TOF sensor. The lens condenses part of the light reflected by the measuring object onto the TOF sensor.

### CROSS-REFERENCE TO RELATED APPLICATIONS

The entire disclosure of Japanese Patent Application No. 2020-106693 filed on Jun. 22, 2020 is incorporated herein by reference in its entirety.

### BACKGROUND

### Technological Field

The present disclosure relates to an intraoral measurement device.

### Description of Related Art

There is known a method of optically measuring the three-dimensional form of a measuring object on the basis of images that capture, from different angles, multiple patterns projected on the measuring target (for example, disclosed in JP2009-165558A). Such a method may involve a measurement error when the relative position or angle between the measuring device and the measuring object changes while imaging the patterns.

When the measuring object is a model, the model can be fastened during the measurement. When the measuring object is an intraoral object of a subject, however, firmly fastening the subject is too stressful for the subject and is therefore not feasible. Further, in measuring teeth, the measurement device has to be moved to measure all the aspects of the teeth, such as the inside and outside of the teeth, upper and lower jaws, and the occlusion of the teeth, which are difficult to measure at one time. The measurement device therefore may not be fastened.

### SUMMARY

The present invention has been conceived in view of the above issues. Objects of the present invention include reducing measurement errors caused by postural changes of the subject and/or the measurement device.

To achieve at least one of the abovementioned objects, according to an aspect of the present invention, there is provided an intraoral measurement device including a measurement optical system that includes: a laser light source; a time-of-flight (TOF) sensor; and a lens, wherein the laser light source irradiates a measuring region including a measuring target with laser light that is intensity-modulated in synchronization with the TOF sensor, and the lens condenses part of the light reflected by the measuring object onto the TOF sensor.

### BR1EF DESCR1PTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention, wherein:

FIG. 1 shows a configuration of an intraoral measurement device in an embodiment;

FIG. 2A shows the optical path of a light emission system of a measurement optical system in the embodiment;

FIG. 2B shows the optical path of the light reception system of the measurement optical system in the embodiment;

FIG. 3A shows the vertical section of the optical path of the light emission system of the measurement optical system in the embodiment;

FIG. 3B shows the horizontal section of the optical path of the light emission system of the measurement optical system in the embodiment;

FIG. 4A shows the vertical section of the optical path of the light reception system of the measurement optical system in the embodiment;

FIG. 4B shows the horizontal section of the optical path of the light reception system of the measurement optical system in the embodiment;

FIG. 5A shows the vertical section of the optical path of the light emission system of the measurement optical system in a modification of the embodiment;

FIG. 5B shows the horizontal section of the optical path of the light emission system of the measurement optical system in the modification;

FIG. 6A shows the vertical section of the optical path of the light reception system of the measurement optical system in the modification of the embodiment;

FIG. 6B shows the horizontal section of the optical path of the light reception system of the measurement optical system in the modification;

FIG. 7 is a schematic depiction of a fine structure of a diffractive optical element that reflects light on its front surface in the modification of the embodiment;

FIG. 8 is a schematic depiction of a fine structure of a diffractive optical element that reflects light on its back surface and that has one diffraction order in the modification of the embodiment; and

FIG. 9 is a schematic depiction of a fine structure of a diffiactive optical element that reflects light on its back surface and that has two diffraction orders in the modification of the embodiment.

### DETAILED DESCR1PTION OF THE EMBODIMENTS

Hereinafter, an embodiment of the present invention is described with reference to the drawings. However, the scope of the present invention is not limited to the disclosed embodiment.

[Configuration of Intraoral Measurement Device]

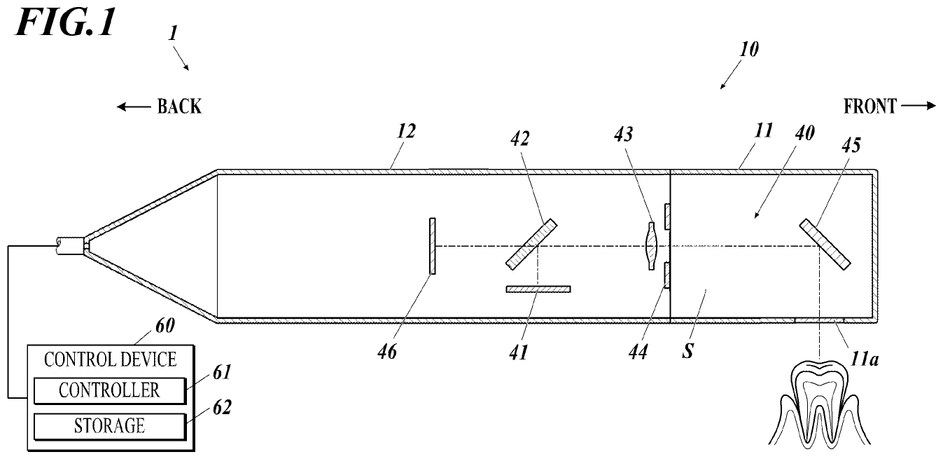

FIG. 1 shows a configuration of an intraoral measurement device 1 in an embodiment. The intraoral measurement device 1 mainly measures the three-dimensional form of the oral cavity/intraoral measuring object of a human body. As shown in FIG. 1, the intraoral measurement device 1 includes a body 10 and a control device 60.

The body 10 is a part to be inserted into the oral cavity. The body 10 houses, in its interior space S, a measurement optical system 40 for measuring the intraoral measuring object.

The measurement optical system 40 includes a laser light source 41, a beam splitter 42, a lens 43, an aperture 44 (first aperture), a minor 45. and a light receiving sensor 46. Among these components, the light receiving sensor 46, the beam splitter 42, the lens 43, the aperture 44, and the mirror 45 are arranged in this order from the back side of the body 10 in the longitudinal direction. The laser light source 41 is positioned at the lateral side of the beam splitter 42. The mirror 45 is positioned at an angle in the front-end part of the body 10 so as to reflect light from the aperture 44 towards the lateral side.

The detailed configuration of the measurement optical system 40 is described later.

The body 10 has a long cylindrical form and consists of a tip part 11 and a base part 12. The tip part 11 is the front part to be first inserted to the oral cavity. The base part 12 is the back part opposite the tip part 11. The tip part 11 houses the mirror 45 of the measurement optical system 40. The base part 12 houses the laser light source 41, the beam splitter 42. the lens 43, the aperture 44, and the light receiving sensor 46 of the measurement optical system 40.

The tip part 11 is detachable from the base part 12. When the tip part 11 is detached from the base part 12, the aperture 44 is exposed at the front end of the base part 12.

The control device 60 is connected to the body 10 and centrally controls the intraoral measurement device 1 in accordance with the user's operation, for example. More specifically, the control device 60 includes a controller 61 and a storage 62.

The storage 62 stores various programs for operating the intraoral measurement device 1 and various kinds of data, such as information obtained by the measurement optical system 40.

The controller 61 controls the operation of the body 10 (measurement optical system 40) to measure the three-dimensional form in the oral cavity in accordance with the programs stored in the storage 62.

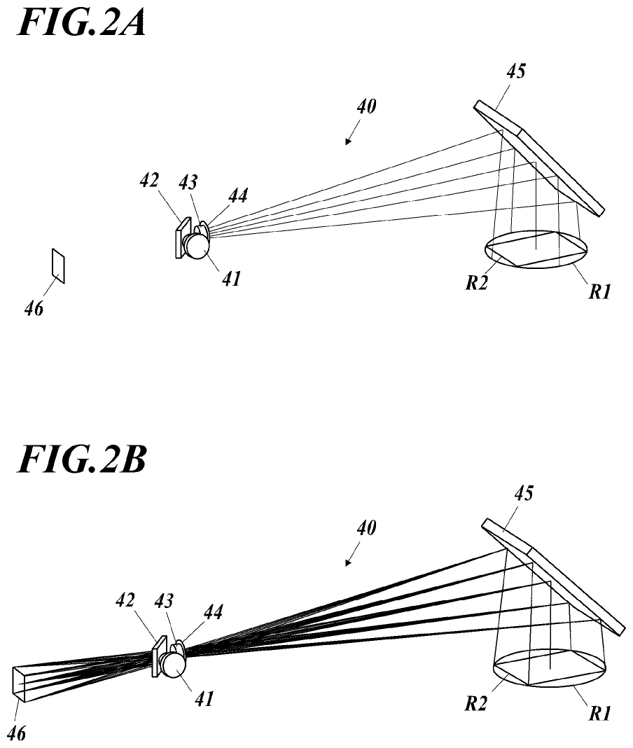

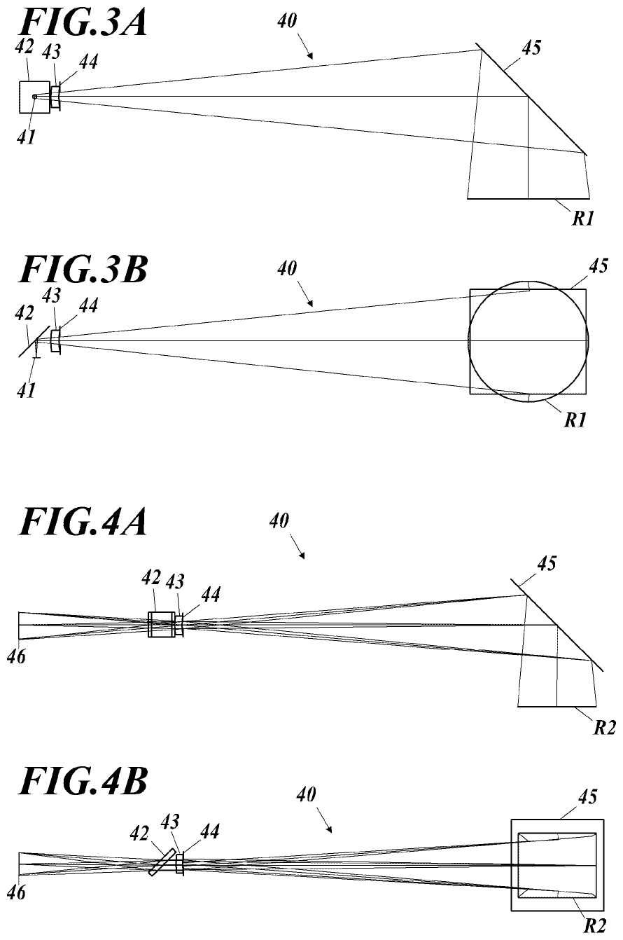

FIG. 2A shows the optical path of the light emission system in the measurement optical system 40. FIG. 2B shows the optical path of the light reception system in the measurement optical system 40. FIG. 3A shows the vertical section of the optical path of the light emission system in the measurement optical system 40. FIG. 3B shows the horizontal section of the optical path of the light emission system in the measurement optical system 40. FIG. 4A shows the vertical section of the optical path of the light reception system in the measurement optical system 40. FIG. 4B shows the horizontal section of the optical path of the light reception system in the measurement optical system 40.

As shown in FIG. 2A, the measurement optical system 40 includes the laser light source 41, the beam splitter 42, the lens 43, the aperture 44, the mirror 45, and the light receiving sensor 46.

The laser light source 41 is a laser diode.

The beam splitter 42 is a polarizing beam splitter.

The lens 43 is placed at a specific position on the optical path with respect to the laser light source 41 and the light receiving sensor 46. More specifically, the lens 43 is placed such that the optical path between the light receiving sensor 46 and the lens 43 is longer than the optical path between the lens 43 and the laser light source 41.

The aperture 44 is a round opening part placed on the optical path between the lens 43 and the measuring region R1.

The mirror 45 is a plane mirror placed on the optical path between the lens 43 and the measuring region R1.

The light receiving sensor 46 is a time-of-flight (TOF) sensor.

As shown in FIGS. 2A, 3A, 3B, the light emission system of the measurement optical system 40 emits light (laser light) from the laser light source 41. The intensity of the laser light is modulated with sine waves and/or square waves by the controller 61 in synchronization with the light receiving sensor 46. The light emitted by the laser light source 41 is reflected by the beam splitter 42 and then condensed by the lens 43. The light condensed by the lens 43 is divergent. The range of angles of the condensed light is narrower than the range of angles of the light before entering the lens 43. The range of angles of light is regulated by the aperture 44 right behind the lens 43. The direction of light is then changed by the mirror 45, so that the light passes through the light passing window 1 la shown in FIG. 1 at the front end of the body 10 (tip part 11) to strike the measuring region R1 in the oral cavity.

In FIG. 2A, the measuring region R1 that is irradiated by the light emission system of the measurement optical system 40 is shown in an oval shape, and only five light rays are shown that pass through the center, the upper edge, the lower edge, the left edge, and the right edge of the aperture 44, respectively.

When the measuring object (e.g., tooth) is in the measuring region R1, the light emitted by the light emission system is diffusively reflected on the surface of the measuring object, as shown in the light reception system of the measurement optical system 40 in FIGS. 2B, 4A, 4B. At least part of the diffusively reflected light enters the body 10 and is reflected by the minor 45 and passes through the aperture 44. The light that has passed through the aperture 44 is condensed by the lens 43, penetrates the beam splitter 42, and is received by the light receiving sensor 46.

The light that has penetrated the beam splitter 42 is received by the light receiving sensor 46. As the beam splitter 42 is a polarizing beam splitter, the light that is regularly reflected by the measuring object (e.g., tooth) maintains its direction of polarization and does not penetrate the beam splitter 42, thereby not being used in the measurement. The light that is diffusively reflected by the measuring object has a disturbed polarization direction. Part of the diffusively reflected light is reflected by the beam splitter 42, whereas other part thereof penetrates the beam splitter 42. The light that has penetrated the beam splitter 42 is used for the measurement.

The controller 61 calculates the difference in phases between the output and input on the basis of information on changes in intensities obtained through time-resolved measurement by the light receiving sensor 46. On the basis of the difference, the controller 61 calculates the distance to the measuring object. The controller 61 thus measures the form of the measuring object in the oral cavity.

FIG. 2B shows only the light-receiving surface of the light receiving sensor 46 for simplification. Further, in FIG. 2B, five light rays that pass through the center, the upper edge, the bottom edge, the left edge, and the right edge of the aperture 44, respectively are shown among light rays that arrive at any of five points (the center and four corners) of the light-receiving surface of the light receiving sensor 46. The light reception system condenses light rays that come from a point of the measuring region R1 onto a point of the light receiving sensor 46. The light reception system in FIG. 2B is therefore different from the light emission system shown in FIG. 2A in that each of the five light rays is shown as a bundle of rays.

The rectangle region (detectable region R2) within the measuring region R1 is a region detectable by the light receiving sensor 46. The actual measuring region R1 has a height in the top-bottom direction in the figure, as the measurement optical system 40 measures the three-dimensional form of the measuring object. The angles of outlying light rays slightly differ between the light emission system and the light reception system. Due to the difference, the measuring region R1, which is the region irradiated with the laser light, may not exactly coincide with the detectable region R2, which is detectable by the light receiving sensor 46. The measuring region R1 should be larger than the detectable region R2. The measuring region R1 and the detectable region R2 may not be in a specific shape.

### Technical Effects of Embodiment

As described above, in this embodiment, the light receiving sensor 46 in the measurement optical system 40 is a TOF sensor.

The measurement optical system 40 therefore instantly measures the three-dimensional form of the measuring object from a certain viewpoint by performing the TOF measurement. This can reduce measurement errors caused by postural changes of the subject and/or the measurement device (body 10) during the measurement.

Further, in this embodiment, the aperture 44 is placed on the optical path between the lens 43 and the measuring region R1.

That is, the aperture 44 is positioned closer to the measuring object (closer to the front end of the body 10) than the lens 43. When, for example, the tip part 11 at the front end is detached for sterilization, the aperture 44 (stop) is the outermost part (exposed part) of the base part 12. Such a configuration can minimize the width of the opening part to avoid stains on the lens 43 and other components.

Further, in this embodiment, the beam splitter 42 causes the light emitted by the laser light source 41 to be incident on the lens 43, and the lens 43 causes the incident light coining from the beam splitter 42 to strike the measuring region R1 in the state of diverging light.

The measuring region R1 can therefore be irradiated efficiently with a smaller amount of laser light. Further, the lens 43 is also used in the light reception system, so that the body 10 can be compact. Further, the light emission system and the light reception system have a common optical path between the lens 43 and the measuring object. Such a configuration can make the front end of the body 10 smaller than a configuration in which the light emission system and the light reception system have different optical paths.

Further, in this embodiment, the optical path between the light receiving sensor 46 and the lens 43 is longer than the optical path between the lens 43 and the laser light source 41.

In such a configuration, the light reception system condenses light coming from a certain point of the measuring object onto a point of the light receiving sensor 46, whereas the light emission system emits light such that light emitted from a certain point of the laser light source 41 diverges to cover the measuring region R1. It is therefore preferable that the optical path between the light receiving sensor 46 and the lens 43 be longer than the optical path between the lens 43 and the laser light source 41.

Further, in this embodiment, the beam splitter 42 reflects at least part of light emitted by the laser light source 41 towards the measuring region R1 and transmits at least part of light reflected by the measuring object towards the light receiving sensor 46.

In such a configuration, the light receiving sensor 46 can be placed farther from the beam splitter 42 on the optical path from the lens 43, as compared with a configuration in which the beam splitter 42 transmits light towards the measuring region R1 and reflects light reflected by the measuring object towards the light receiving sensor 46 (i.e., transmission and reflection by the beam splitter 42 are reversed). Accordingly, the body 10 can be more compact.

Further, in this embodiment, the beam splitter 42 is a polarizing beam splitter. The polarizing beam splitter can improve efficiency as compared with a half mirror having 50% reflectivity.

In measuring a tooth, strong regularly-reflected light may occur when the surface of the tooth is wet. When the normal of part of the tooth coincides with the direction of the sight of the measurement device, the part looks brighter than the surrounding parts. This may affect the measurement. The polarizing beam splitter transmits approximately half of scattered light that has disturbed polarization towards the light receiving sensor 46, while restraining regularly-reflected light that maintains its polarization direction from entering the light receiving sensor 46.

Further, in this embodiment, the mirror 45 is placed on the optical path between the lens 43 and the measuring region R1.

As a tooth has an uneven surface, part of the tooth may be invisible in shadow when seen at an angle in the measurement. The tooth therefore needs to be measured by changing the posture of the measurement device such that the device faces each part of the tooth. When the components of the measurement optical system 40 are arranged in a straight line from the measuring region R1 to the light receiving sensor 46, the front end of the measurement device becomes larger, and the subject feels more stress.

In this embodiment, the mirror 45 is placed at the front end of the body 10 such that the laser light from the lens 43 strikes the tooth via the mirror 45 and that the light reflected by the tooth is reflected by the same mirror 45 towards the lens 43 and guided to the light receiving sensor 46. Such a configuration can make the front end of the body 10 more compact as compared with a configuration in which the components from the lens 43 to the measuring region R1 are arranged in a straight line.

Further, the mirror 45 is a simple plane mirror. Even when the front end part of the body 10 (tip part 11) including the mirror 45 is sterilized, the sterilization less affects the optical performance of the measurement device.

[Modification]

A measurement optical system 40A in a modification of the embodiment is described. Hereinafter, differences between the modification and the above embodiment are mainly described. The components in the modification that are the same as the components in the above embodiment are denoted by the same reference numerals, and detailed description thereof is omitted.

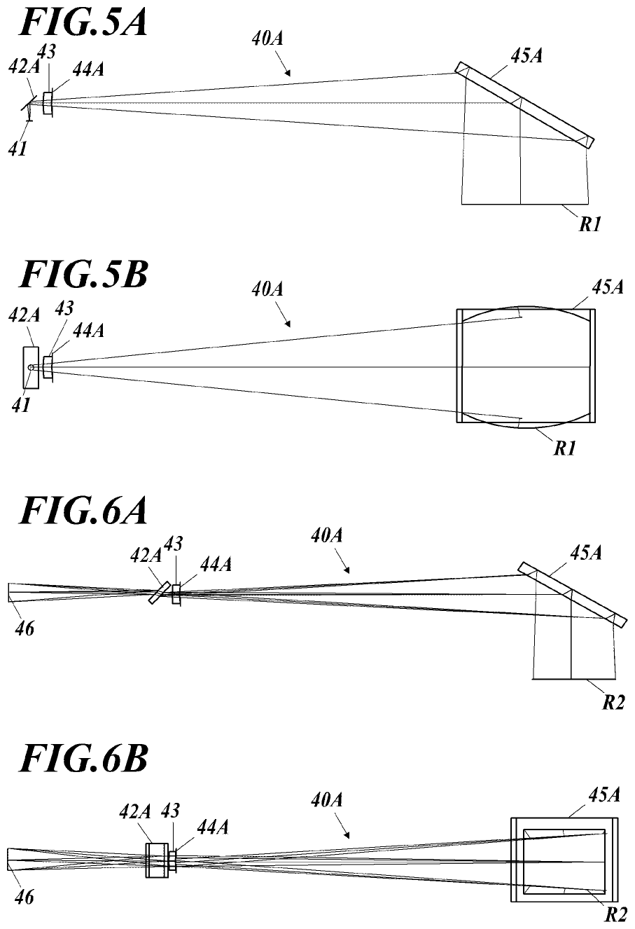

FIG. 5A shows the vertical section of the optical path of the light emission system in the measurement optical system 40A. FIG. 5B shows the horizontal section of the optical path of the light emission system in the measurement optical system 40A. FIG. 6A shows the vertical section of the optical path of the light reception system in the measurement optical system 40A. FIG. 6B shows the horizontal section of the optical path of the light reception system in the measurement optical system 40A.

As shown in FIGs.5A, 5B, 6A, 6B, the measurement optical system 40A in this modification includes a beam splitter 42A, an aperture 44A, and a mirror 45A instead of the beam splitter 42, the aperture 44, and the mirror 45 in the above embodiment. The other components of the measurement optical system 40A are the same as those of the measurement optical system 40 in the above embodiment. However, the laser light source 41 in the modification is positioned below the beam splitter 42A.

This is because, in the modification, the optical path of both the light emission system and the light reception system is wider in the horizontal direction than in the vertical direction around the beam splitter 42A. On the other hand, in the above embodiment, the optical path of the light emission system has the same width in the vertical and horizontal directions, whereas the optical path of the light reception system is wider in the vertical direction than in the horizontal direction. The laser light source 41 is therefore provided on the lateral side of the beam splitter 42 in the above embodiment.

The mirror 45A is a diffractive optical element and, in this modification, is a diffractive optical element that reflects light on its back surface (back-reflective diffractive optical element). The front surface of the minor 45A that faces the lens 43 (the lower-left surface in FIG. 5A) is a plane surface that penetrates light, and the back surface of the mirror 45A (the upper-right surface in FIG. 5A) is a surface that diffracts and reflects light.

The mirror 45A in FIG. 5A leans in the counterclockwise direction as compared with the mirror 45 in the above embodiment. The mirror 45A, which is the diffractive optical element, reflects light rays that enter the center of the mirror 45A straight towards the measuring region R1. The size of the measuring region R1 is substantially the same as that in the above embodiment.

The mirror 45A, which leans more than the minor 45 in the above embodiment, takes up less space in the vertical direction than the mirror 45. This allows the body 10 to have a narrower front end part while keeping the measuring region R1 at around the same size, and therefore can reduce stress on the subject.

In other words, the measuring region R1 can be widened by using the mirror 45A as the diffractive optical element and by setting the width of the front end part of the body 10 to be approximately as wide as that in the above embodiment (i.e., setting the angle of the mirror 45A such that the width of the front end part of the body 10 is approximately as wide as that in the above embodiment). The wider measuring region R1 allows a wider region to be measured at one time, and accordingly shortens time required for measurement. Further, the wider measuring region R1 is advantageous in terms of accuracy in measurement. For example, in measuring teeth some of which are missing, the soft part between the teeth may not be accurately measured. In the case, measuring two separate teeth in one sight can improve the accuracy.

The fine structure of the mirror 45A as the diffractive optical element is described.

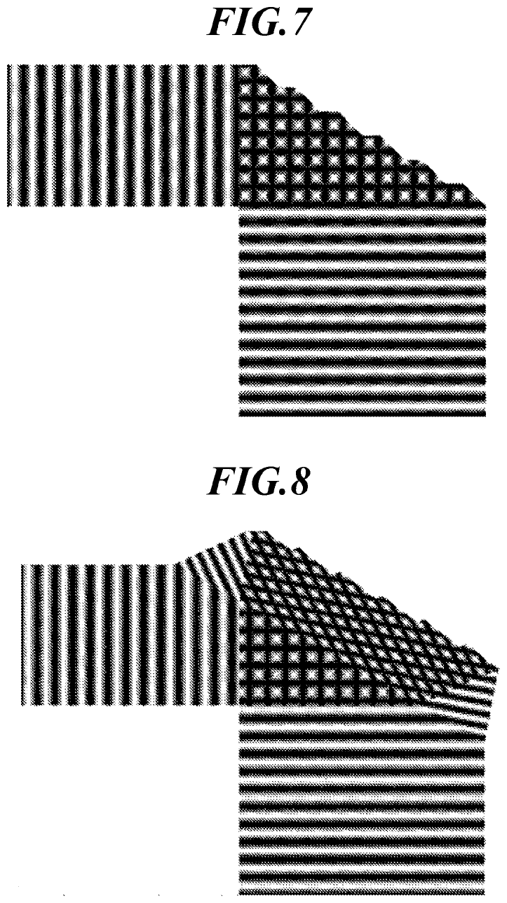

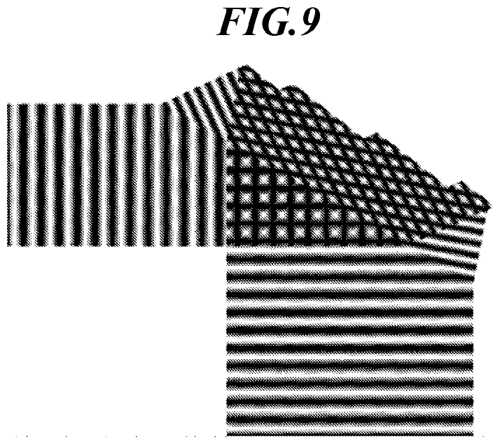

FIGS. 7, 8, 9 schematically depict examples of the fine structure of the diffractive optical element. FIG. 7 shows a diffractive optical element that reflects and diffracts light on its front surface. FIG. 8 shows a diffractive optical element that reflects and diffracts light on its back surface in one diffraction order. FIG. 9 shows a diffractive optical element that reflects and diffracts light on its back surface in two diffraction orders. In FIG. 7, the upper-right stepped surface corresponds to the front surface of the mirror 45A. In FIGS. 8, 9, the upper-right stepped surface corresponds to the back surface of the mirror 45A. These figures show the vertical section of the mirror 45A and light, where the wavelength is shown extra long and the width and depth of the grooves are increased in proportion to the wavelength. The light is shown as narrow parallel light in a form of waves.

As shown in FIGS. 7, 8, 9, grooves are cut at regular intervals in the front/back surface of the mirror 45A.

The grooves are formed linearly in a direction perpendicular to the paper's surface so as to have a uniform section. The diffractive optical element that has linear grooves at regular intervals does not have refractive power. The fine structure has a saw-toothed shape the height of which is approximately the same as the wavelength. The light before being incident on the diffractive optical element is a horizontal wave. The light after being reflected by the diffractive optical element is a vertical wave.

As shown in FIG. 7, the mirror 45A may be a diffractive optical element that reflects and diffracts light on its front surface (front-reflective diffractive optical element). In the case, each groove in the diffractive surface consists of a surface that extends in the paper's right-left direction and a surface angled at 45 degrees. The angled surface is an effective optical surface.

As shown in FIG. 8, the mirror 45A may be a diffractive optical element that reflects and diffracts light on its back surface (back-reflective diffractive optical element) in one diffraction order. In the case, one groove shifts the phase of light waves by one wavelength. The interval between grooves in FIG. 8 is the same as that of the front-reflective diffractive optical element in FIG. 7, whereas the saw-toothed shape in FIG. 8 is different from that in FIG. 7. As with the front-reflective diffractive optical element, the diffractive surface of the back-reflective diffractive optical element has effective optical surfaces and ineffective walls. On the other hand, the back-reflective diffractive optical element has wider effective optical surfaces and is therefore more efficient. Further, the back-reflective diffractive optical element has deeper grooves in the direction orthogonal to the envelope than the front-reflective diffractive optical element. The back-reflective diffractive optical element is therefore easier to form.

As shown in FIG. 9, the mirror 45A may be a diffractive optical element that reflects and diffracts light on its back surface (back-reflective diffractive optical element) in two diffraction orders. In the case, one groove shifts the phase of light waves by two wavelengths. The diffractive optical element with two diffraction orders therefore has: the fine structure that is twice as large as the fine structure of the element with one diffraction order; and grooves that are twice as wide and deep as the grooves of the element with one diffraction order. Wider grooves are easier to form and increases diffraction efficiency.

The mirror 45A may be a diffractive optical element that reflects and diffracts light on its back surface (back-reflective diffractive optical element) in two or more diffraction orders.

As shown in FIGS. 5A, 5B, after passing through the aperture 44A, light has different ranges of angles in the vertical direction and in the horizontal direction. The aperture 44A has an opening part in a shape of a circle the upper and bottom parts of which are linearly cut off (i.e., oval-shaped opening part). The measuring region R1 has a distorted oval shape in the right-left direction in FIG. 5B due to the distortion caused by the diffractive optical element (mirror 45A).

As shown in FIGS. 6A, 6B, the surface of the beam splitter 42A that faces the light receiving sensor 46 (the upper-left surface in FIG. 6A) regulates only the width in the vertical direction of light that penetrates the beam splitter 42A, the vertical direction being orthogonal to the direction of the grooves of the diffractive optical element (mirror 45A). The surface of the beam splitter 42A that faces the light receiving sensor 46 is an example of the second aperture in the present invention. With the second aperture, the light reception system regulates the width of light only in the vertical direction, so that the optical path is narrowed.

This is to increase the depth of field in the light reception system. The image surface in the vertical section deviates from the image surface in the horizontal section owing to a side effect of the diffractive optical element (mirror 45A). The optical path therefore needs to be narrowed in order to increase the depth of field. On the other hand, the diffractive optical element does not affect the horizontal width of light. The horizontal section is therefore similar to that in the above embodiment. That is, the width of light is regulated by the aperture 44A.

The second aperture may be an individual aperture that is separate from the beam splitter 42A and that is positioned slightly closer to the light receiving sensor 46 than the beam splitter 42A. In the case, the beam splitter 42A may be configured the same as the beam splitter 42 in the above embodiment.

As described above, according to this modification, the minor 45A is a reflective diffractive optical element and therefore can have optical functions. For example, the measuring region R1 can be widened without increasing the width of the front-end part of the body 10 or can be kept at approximately the same size while decreasing the width of the front-end part of the body 10.

Further, according to this modification, the mirror 45A as the reflective diffractive optical element has linear grooves formed side by side at regular intervals.

This allows the mirror 45A to be a diffractive optical element that does not have refractive power. Such a diffractive optical element does not condense light but can change the angle of light. The diffractive optical element can therefore provide the measurement device with a desirable optical function of expanding the field of view without increasing the width of the body 10. The above-described diffractive optical element is also easier to produce than a diffractive optical element having refractive power. The above-described diffractive optical element is therefore advantageous in a case of producing the diffractive optical element out of material resistant to high temperature in sterilization, such as glass. The leading-end part of the body 10 (tip part 11) may be a single-use tip made of material vulnerable to high temperature, such as resin. In such a case, the above-described diffractive optical element without refractive power can be produced at relatively low cost with a smaller amount of variation.

Further, according to this modification, the second aperture (the surface of the beam splitter 42A that faces the light receiving sensor 46) is positioned on the optical path between the beam splitter 42A and the light receiving sensor 46.

The mirror 45A may not function the same way in the direction of diffractive effects in the light emission system and the light reception system, because the mirror 45A without refractive power still shifts the image forming surface owing to its diffractive effects. To deal with the above issue, the second aperture that is effective only in the light reception system is used to increase the depth of field. Further, when a diffractive optical element is used in the time-of-flight method, some light rays may have different optical path lengths. The wider the light is when diffracted by the diffractive optical element, the light coming from a point of the measuring object and eventually passing through the aperture 44A, the lower the accuracy is in measuring the distance. It is therefore preferable to use the second aperture to narrow the width of light in the direction of diffractive effects. Further, when the lens 43 is used in both the light emission system and the light reception system, it is preferable to position the second aperture on the optical path between the beam splitter 42A and the light receiving sensor 46.

Further, according to this modification, the second aperture (the surface of the beam splitter 42A that faces the light receiving sensor 46) regulates the width of light only in a direction orthogonal to the direction in which the grooves in the mirror 45A extend.

Shift of the image surface occurs only in the direction in which the minor 45A (diffractive optical element) widens the width of light. It is therefore preferable that the second aperture regulate the width of beam only in a direction orthogonal to the direction of the grooves.

Further, according to this modification, the mirror 45A is a back-reflective diffractive optical element that reflects light on its back surface.

Types of reflective diffractive optical element include: a front-reflective diffractive optical element that reflects and diffracts light on its front surface; and a back-reflective diffractive optical element that allows light to penetrate its front surface and that reflects and diffracts the light on its back surface. The back-reflective diffractive optical element can have shallower grooves. According to this modification, the width of light is expanded by adjusting the angle of the mirror 45A. In such a case, the back-reflective diffractive optical element can reduce a difference in angles between the incident light and the reflected light and can restrain shielding by the walls.

Further, according to this modification, the mirror 45A is a reflective diffractive optical element having two or more diffraction orders.

The narrower the interval between grooves is, the stronger the diffraction effect of the diffractive optical element is. The narrower interval, however, relatively increases effects of manufacturing errors. Even if no manufacturing error is present, the diffraction efficiency decreases as the interval between grooves is closer to the wavelength. To deal with this, the diffractive optical element is formed to have two or more diffraction orders so that one groove shifts the phase of light waves by two wavelengths or more. Accordingly, the interval between grooves can be widened in proportion to the diffraction order. Although the depth of grooves is also increased in proportion to the diffraction order, it is preferable to widen the interval when the interval is too narrow.

[Others]

The above-described embodiment and modifications of the present invention do not limit embodiments to which the present invention is applicable, and can be appropriately modified without departing from the scope of the present invention.

Although the embodiment of the present invention has been described and illustrated in detail, the disclosed embodiment is made for purposes of illustration and example only and not limitation. The scope of the present invention should be interpreted by terms of the appended claims.

What is claimed is:

1. An intraoral measurement device comprising a measurement optical system that includes:
a laser light source;
a time-of-flight (TOF) sensor; and
a lens, wherein
the laser light source irradiates a measuring region including a measuring target with laser light that is intensity-modulated in synchronization with the TOF sensor, and
the lens condenses part of the light reflected by the measuring object onto the TOF sensor.

2. The intraoral measurement device according to claim 1, wherein
the measurement optical system further includes a first aperture on an optical path between the lens and the measuring region.

3. The intraoral measurement device according to claim 1, wherein
the measurement optical system further includes a beam splitter that causes the light emitted by the laser light source to be incident on the lens, and
the lens causes the incident light coining from the beam splitter to strike the measuring region in a state of diverging light.

4. The intraoral measurement device according to claim 3, wherein
an optical path between the TOF sensor and the lens is longer than an optical path between the lens and the laser light source.

5. The intraoral measurement device according to claim 3, wherein the beam splitter reflects at least part of the light emitted by the laser light source towards the measuring region, and transmits at least part of the light reflected by the measuring object towards the TOF sensor.

6. The intraoral measurement device according to claim 3, wherein the beam splitter is a polarizing beam splitter.

7. The intraoral measurement device according to claim 3, wherein the measurement optical system further includes a mirror placed on an optical path between the lens and the measuring region.

8. The intraoral measurement device according to claim 7, wherein the mirror is a plane mirror.

9. The intraoral measurement device according to claim 7, wherein the mirror is a reflective diffractive optical element.

10. The intraoral measurement device according to claim 9, wherein the reflective diffractive optical element has linear grooves formed side by side at regular intervals.

11. The intraoral measurement device according to claim 9, wherein the measurement optical system further includes a second aperture placed on an optical path between the beam splitter and the TOF sensor.

12. The intraoral measurement device according to claim 11, wherein
the reflective diffractive optical element has linear grooves formed side by side at regular intervals, and
the second aperture regulates a width of the light only in a direction orthogonal to a direction in which the grooves extend.

13. The intraoral measurement device according to claim 9, wherein the reflective diffractive optical element reflects the light on a back surface.

14. The intraoral measurement device according to claim 9, wherein the reflective diffractive optical element has two or more diffraction orders.
